(19) United States
(12) Patent Application Publication
(10) Pub. No.: US 2025/0134449 A1
(43) Pub. Date: May 1, 2025

(54) SMART-CLOTHES - FABRIC-BASED MICROFLUIDIC SENSORS FOR WEARABLE HEALTH MONITORING

(71) Applicant: UNIVERSITY OF MARYLAND, BALTIMORE COUNTY, Baltimore, MD (US)

(72) Inventors: Chengpeng CHEN, Columbia, MD (US); Tao ZHANG, Columbia, MD (US); Adam Michael RATAJCZAK, Abingdon, MD (US)

(21) Appl. No.: 18/921,076

(22) Filed: Oct. 21, 2024

Related U.S. Application Data
(60) Provisional application No. 63/593,003, filed on Oct. 25, 2023.

(51) Int. Cl. A61B 5/00; A41D 1/00; A61B 5/1477; A61B 10/00; D06M 15/233; D06M 15/31; D06M 23/10; D06M 23/16; D06M 101/06

(52) U.S. Cl. CPC A61B 5/4266; A41D 1/002; A61B 5/1477; A61B 10/0064; D06M 15/233; D06M 15/31; D06M 23/10; D06M 23/16; D06M 2101/06; D06M 2200/12

(57) ABSTRACT

Fabric-based microfluidics, as a part of an article of clothing, is described herein. Advantageously, the fabric-based microfluidics, based on the infusion of a polymer such as acrylonitrile butadiene styrene (ABS) films through fabrics to form hydrophobic areas, are simple to make, robust, and suitable for efficient sweat delivery. Electrodes can be screen-printed onto the fabric-based microfluidic. Coupled with a low-cost, wearable potentiometer capable of wireless data transfer, [Ca²⁺] or other analyte species in a wearer's sweat can be quantified. Advantageously, regular articles of clothing can be turned into biochemically smart platforms for health monitoring, which will broadly benefit human healthcare.

### CROSS REFERENCE TO RELATED APPLICATIONS

This application claims priority to U.S. Provisional Patent Application No. 63/593,003 filed on Oct. 25, 2023 in the name of Chengpeng CHEN et al. entitled “STEP FORWARD FOR SMART CLOTHES—FABRIC BASED MICROFLUIDIC SENSORS FOR WEARABLE HEALTH MONITORING,” which is hereby incorporated by reference herein in its entirety.

### FIELD OF THE INVENTION

This invention relates to methods of making and using fabric-based microfluidics for wearable sensing of analyte species.

### BACKGROUND OF THE INVENTION

Wearable biochemical sensing is significant because of its capability for continuous health monitoring in a real-time and noninvasive manner. There are various biofluids that can be targeted for biomarker screening, including blood, tears, urine, and saliva. Nonetheless, blood is difficult to access for noninvasive and continuous detections; the on-and-off nature of tears is not suitable for immediate real-time monitoring, and neither is urine; while saliva is continuously produced, it can be dramatically affected by food or drinks. Therefore, sweat has recently gained great attention and interest in both academic and industrial research as an ideal biofluid for continuous health monitoring (Bandodkar, 2014; Ghaffari, 2021; Xiao, 2022; Moonen, 2020; Yang, 2022). Sweat is secreted continuously on body surface—around 3 μL/min with inaction and over 10 μL/min when sporting for adults per square centimeter (Brueck, 2018; Emaminejad, 2017). Moreover, sweat contains a rich library of chemicals, such as metabolites, proteins, hormones, and ions, which can provide extensive physiological insights (Anastasova, 201⁷; Sim, 2022; Wang, 2021). The concentrations of the chemicals in sweat are also found correlated with their blood levels (Sempionatto, 2021).

Since 2016, when Roger's group first reported a wearable colorimetric microfluidic sensor (Koh, 2016), various wearable sensor platforms have been reported, which can be generally categorized into two groups: static and microfluidic. Static sensors apply a substrate as an exposed detection area directly attached onto human skin to measure the target analytes (Gao, 2016). A typical example is a flexible polyethylene terephthalate patch with screen-printed electrodes, which can be taped or fastened on skin for biochemical analyses in sweat (Gao, 2016; Wang, 2019; Zhang, 2020). Although the setup is simple and straightforward, sample turnover can be a notable problem—accumulated molecules from sweat at the detection area may not be refreshed efficiently, compromising the temporal resolution for effective continuous measurements. Microfluidic models, on the other hand, apply fluidic features for active sweat delivery through the detection zone (e.g., electrochemistry or colorimetry) via nature pressure (˜70 kPa) produced from the dermal duct of the sweating pores and capillary forces (Bariya, 2018).

Currently, most reported microfluidic wearable sensor platforms are made from soft lithography based on polydimethylsiloxane (PDMS) (Raj, 2020; Akther, 2020; K Almeida Monteiro Melo Ferraz, 2020; Morbioli, 2020). Nonetheless, such devices require well-trained personnel and extensive facilities to be fabricated, applied, and maintained, which can prevent broad adoption of the technology from technical, costly, and applicable perspectives. New microfluidic platforms that are simple to produce, robust, comfortably wearable, allowing detector integration, and can effectively deliver sweat will be critical for wearable biochemical sensing applications.

Paper-based 2D microfluidics, which utilize capillary actions to drive liquid flow within channels defined by hydrophobic barriers, show some promise (Martinez, 2008: Martinez, 2010; Dungchai, 2009: Cincotto, 2019; Liu, J., 2020; Liu, P., 2020). Due to its simplicity, wax printing has been the prevailing method to create hydrophobic barriers through paper substrates (Altundemir, 2017; Dungchai, 2011; Nilghaz, 2019). However, it is challenging to apply a paper-based microfluidic for sweat analyses on human body surfaces because paper loses its integrity after being soaked.

The goal therefore is to create hydrophobic barriers through fabrics to define microfluidic channels and reservoirs. A few papers have shown successful wax-printing of microfluidics on fabrics (Nilghaz, 2019; Liu, 2015). However, the applications were not different from paper-based apparatus (just switching paper by fabrics for similar applications) (Vatansever, 2012; Farajikhah, 2019). The suitability of such apparatus for wearable sensing has not been tested. While proprietary, the wax used to print paper-based microfluidics is mainly paraffin-based (Prabhu, 2020). It is challenging to wax-print microfluidics on a wearable device because paraffin starts to soften and melt above 35° C., causing ever-changing microfluidic structures and uncomfortable wearing experiences (due to the melted wax). Soft lithography is another way to make paper-based microfluidics by infusing photopolymers (e.g., SU-8) through a substrate, followed by photomasking to cure the polymer in the predesigned hydrophobic areas (Urbanski, 2006; Carrilho. 2009; Zhang, 2022). However, this method takes many steps and utilizes a large amount of organic solvents, and hence generates an enormous amount of waste.

Accordingly, there continues to be a need in the art for a new method of producing fabric-based microfluidics for wearable sensing. The technology should be highly reproducible for precisely controlled microchannels and reservoirs, with the capability of detector integration, and simple and scalable so that it is translational to broadly benefit wearable sensor developments.

### SUMMARY OF THE INVENTION

In some aspects, an article of fabric comprising a hydrophobic microfluidic pattern thereon is described, wherein the pattern comprises at least one reservoir and a reservoir channel connected to the reservoir, and wherein the hydrophobic microfluidic pattern comprises a polymer.

In some other aspect, a method of introducing a microfluidic pattern to fabric is described, said method comprising:
introducing a pattern onto a film comprising a polymer;
positioning the film onto a first side of the fabric to produce a polymer film-fabric stack; and
exposing the polymer film-fabric stack to a solvent vapor to dissolve the polymer, wherein the dissolved polymer diffuses into the fabric to form hydrophobic areas in and/or on the fabric that define the microfluidic pattern.

In some other aspects, a wearable sensing device is described, said device comprising: an article of fabric comprising a hydrophobic microfluidic pattern thereon, wherein the pattern comprises at least one reservoir and a reservoir channel connected to the reservoir, wherein the hydrophobic microfluidic pattern comprises a polymer, and wherein each reservoir comprises an electrode: an electronics module; and a control module, wherein the electrodes, the electronics module and the control module are communicatively connected to one another.

In still other aspects, a method of quantitating an analyte species found in sweat of a subject in real time, said method comprising:
positioning at least one indicator electrode in a first reservoir and a reference electrode in a second reservoir of an article of fabric comprising a hydrophobic microfluidic pattern thereon, wherein the pattern comprises at least two reservoirs and a reservoir channel connected to each reservoir, and wherein the hydrophobic microfluidic pattern comprises a polymer;
electrically connecting the electrodes to a potentiometer; and
receiving signals from the potentiometer as sweat contacts the electrodes, wherein an electronic response of the potentiometer changes as sweat contacts the electrodes; and
quantitating the amount of analyte species found in the sweat of the subject based on the signals received from the potentiometer.

Other aspects and advantages will be more fully apparent from the ensuing disclosure and appended claims.

### BRIEF DESCRIPTION OF THE FIGURES

FIG. 1A illustrates the fabrication process to make fabric-based microfluidics.

FIG. 1B is an image of the front of the laser-cut ABS film.

FIG. 1C is an image of blue dye filling the reservoir and channel (Scale bar=5 mm).

FIG. 1D illustrates an embodiment of an optimized reservoir shape and channel width (Scale bar=1 mm), comprising two reservoirs.

FIG. 1E illustrates an embodiment of an optimized reservoir shape comprising three reservoirs.

FIG. 2 illustrates electrodes screen-printed on the fabric-based microfluidics. The electrodes on the side reservoirs were identical indicator electrodes for duplicate measurements in each experiment, and the electrode in the middle reservoir was a Ag/AgCl reference electrode.

FIG. 3A is an example of ion chromatography to detect [Ca²⁺] in a standard Ca²⁺ solution (0.5 mM) for result validation.

FIG. 3B is an example of ion chromatography to detect [Ca²⁺] in a sweat sample (10 μL) spiked in the standard Ca²⁺ solution (5 mL) for result validation.

FIG. 4A illustrates leakage percentage as a function of the acetone treatment time. (N=5, error bars=standard deviation).

FIG. 4B illustrates channel widths after the various acetone treatment times (designed as 800 m). (N=5, error bars=standard deviation).

FIG. 4C illustrates relative standard deviations of widths at different locations of the same channel after the different acetone treatment times.

FIG. 5A illustrates the channel width in CAD design (squares), ABS pattern after laser cutting (circle), and actual microfluidics on cotton fabric (triangle) (N=5, error=standard deviation).

FIG. 5B illustrates the channel widths before and after 37° C. incubation (N=5, error bar=standard deviation).

FIG. 6A illustrates that with a larger channel width, faster drainage was observed, which then plateaued after 800 μm (N=5, error=standard deviation).

FIG. 6B illustrates the percentage of the lowest-velocity areas (<15% of gross average) in the different shapes.

FIG. 7A illustrates residue dye in the reservoirs as a function of time at a flow rate of 3 L/min. Error bars=standard deviation.

FIG. 7B illustrates residue dye in the reservoirs as a function of time at a flow rate of 5 L/min. Error bars=standard deviation.

FIG. 7C illustrates residue dye in the reservoirs as a function of time at a flow rate of 10 L/min. Error bars=standard deviation.

FIG. 8 is an image of a laser-cut ABS pattern for simultaneous multidevice fabrication. (Scale bar=10 mm).

FIG. 9A illustrates the responses of Ca²⁺ standards on the sensor.

FIG. 9B illustrates a calibration curve of Ca²⁺ responses (voltage vs pCa).

FIG. 9C illustrates selectivity tests of the Ca²⁺ electrodes.

FIG. 9D illustrates accuracy tests of the Ca²⁺ sensor. (N=3, error bar=standard deviation).

FIG. 10A is an image of the setup for Ca²⁺ measurements using the wearable fabric-based microfluidic sensor.

FIG. 10B illustrates detected calcium amounts in sweat (data within 2 min were averaged) by two sensors (open circles), and by the validation method of ion chromatography (closed squares). (n.d.=no significant difference, error bar=standard error of mean).

FIG. 10C illustrates detected calcium concentrations using the same electrodes on the same fabric but without the microfluidic feature (open circles). Significant discrepancies were observed from the validated data (closed squares), indicating the necessity of microfluidics to refresh sweat in the detection zone. (Error bar=standard error of mean).

FIG. 11 illustrates a proposed embodiment of wearable clothes comprising fabric-based microfluidic sensors described herein.

FIG. 12A illustrates an embodiment of the fabric-based microfluidic sensor as a module of a T-shirt, wherein the sensor is integrated into a laser cut square and a potentiometer is integrated to the fabric-based microfluidics.

FIG. 12B illustrates near real-time measurements of [Ca²⁺] with the T-shirt sensor. Error bar=standard error of mean.

The features and advantages of the invention are more fully illustrated by the following non-limiting example, wherein all components are used in a particular form to demonstrate the usability and practice.

### DETAILED DESCRIPTION, AND PREFERRED EMBODIMENTS THEREOF

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The present disclosure relates broadly to wearable microfluidic sensors for quantifying a concentration of a species of interest in a user/wearers sweat. The new technology uses a polymer, such as acrylonitrile butadiene styrene (ABS), film “printing” to create microfluidic hydrophobic barriers through fabrics for robust and precise microfluidic development. The fabric-based microfluidic can further comprise electrodes, which can be connected to a potentiometer for data collection, recording, processing and wireless transfer.

As defined herein, a “fabric” includes, but is not limited to, cotton, bamboo, polyester, linen, lyocell, modal, silk (natural, artificial, or synthetic spider), microfiber, satin, cellulose acetate, cellulose triacetate, rayon, polyester, nylon, viscose, hemp, wool, polypropylene, carbon fibers (such as KEVLAR®), lycra-spandex, thermoplastic polyurethane (TPU), polyamide, polyethylene (PE), polyethersulfone (PES), polyether-polyurea copolymer (such as ELASTAN®), or a blend of two or more of these materials. The fabric can be woven, knitted or nonwoven. In some embodiments, the fabric comprises carbon.

As used herein, the “indictor electrode” is an electrode that is responsive to an analyte species. Any known “ion-selective electrodes (ISEs)” can be considered as one type of indicator electrode. In some embodiments, an indicator electrode comprises an electron conductor as the substrate electrode and an “ion-sensitive phase.” The electron conductor can be of any shape including, but not limited to, wire, rod, needle, array, and planar layer. The possible material of the electron conductor includes but not limited to metal, carbon-based materials, semiconductor, and conductive polymers. The “ion-sensitive phase” is a phase that exhibits electrochemical response toward ionic analyte species. The ion-sensitive phase can be made of any materials that are not fully miscible with water. Representative example materials include plasticizer, plasticizer mixed with nanoparticles, plasticizer mixed with polymers, water-immiscible organic solvent, water-immiscible organic solvent mixed with polymers, silicone rubber, photocured polymer, and polymethacrylate. Example configurations of the ion-sensitive phase include deposited membrane and segment of organic solvent, plasticizer, polymer, or organic gel. In some embodiments, the ion-sensitive phase can be directly coated onto the electron conductor. In some embodiments, there can also be an ion-to-electron transduction layer between the electron conductor and the ion-selective membrane to provide better and stable contact. In various embodiments, the ion-sensitive phase contains ionophore as the recognition element for ionic analyte species. Any uncharged or charged molecules that bind analyte ions can be used as an ionophore. Preferred analyte species ionophores, according to various embodiments, can be hydrophobic ionophores that will stay within the ion-selective phase without significant leaching into the sample. Examples of ionophores that may be used include, but are not limited to, hydrogen ionophore I, valinomycin, nonactin, calcium ionophore I, calcium ionophore II, calcium ionophore IV, potassium ionophore I, potassium ionophore III, sodium ionophore X, sodium ionophore VI, magnesium ionophore I, magnesium ionophore III, magnesium ionophore IV, nitrate ionophore VI, nitrite ionophore I, nitrite ionophore VI, and mercuracarborand. In some embodiments, the amount of ionophore can be, for example, from 0.1 to 20 wt %, preferably from 0.5 to 2 wt % in the ion-sensitive phase.

As used herein, the “reference electrode” is an electrode that is not responsive to an analyte species to a significant extent and provides a relatively constant potential. Various known reference electrodes developed in the area of electrochemistry may be used here. In some embodiments, the reference electrode comprises an electron conductor covered by a reference membrane. Example reference electrodes can include at least three types. First, Ag/AgCl as the electron conductor can be immersed in a solution or hydrogel containing one or more chloride salts. The salt can be fully dissolved or oversaturated. Second, Ag or Ag/AgCl as the electron conductor can be covered by a polymer containing one or more chloride salts. In an embodiment, the salt is not necessarily fully dissolved. Third, a metal or carbon-based electron conductors can be covered by, or positioned in direct contact with, a water-immiscible solvent, plasticizer, polymer, ink, or their combinations containing hydrophobic salts (the so-called “liquid-junction-free” reference electrodes). Overall, representative examples of the electron conductor for the reference electrode include silver coated with silver chloride, metal, carbon-based materials, semiconductors, and conductive polymers. The electron conductor can be of various shapes including, but not limited to, wire, rod, needle, array, and planar layer. Example materials to make the reference membrane can include hydrogel, ink, plasticizer, plasticizer mixed with nanoparticles, plasticizer mixed with polymers, silicone rubber, photocured polymer, polymethacrylate, polyvinyl butyrate, polyvinyl chloride, polyvinyl acetate, and/or ionic liquid. The reference membrane can be a deposited membrane or a segment of solution/material, among other possible geometries. In an embodiment, the reference membrane can contain chemicals that aid in establishing a relatively constant and sample-independent potential. Representative examples of chemicals for the reference membrane include, but are not limited to, inorganic salts, ionic liquid, and hydrophobic salts. In one or more embodiments, preferred inorganic salts can include, for example, chloride salts.

Broadly, the present disclosure relates to the development of microfluidics on fabrics, e.g., as part of an clothing, for the collection and delivery of sweat, via the microfluidic structures, to a sensor platform for measurement of a parameter (e.g., the concentration of an analyte species). For example, a wearable sensing device comprising the microfluidic structures can be a standalone system or as a module of clothing, e.g., a t-shirt, to quantify a species such as [Ca²⁺] in a wearer's sweat. Advantageously, using the microfluidic structures described herein, regular clothing or accessories can then be turned into biochemically smart platforms for health monitoring.

The wearable sensing device described herein allows for non-invasive and real-time measurement of analyte species found in sweat. In some embodiments, measurements can be wirelessly transmitted from the wearable sensing device to a mobile computing device (e.g., a smartphone) using a specialized electronics module containing a multiplexer to facilitate data transmissions from multiple sensors (e.g., via Bluetooth). In some embodiments, a variable data rate is used, thereby allowing the control of which sensors are activated at a given time, extending the battery lifetime of the device and allowing for a smaller form factor (as battery size generally dictates form factor).

In some embodiments, the microfluidic structure of the wearable sensing device is affixed directly to a user's skin, e.g., using tape or glue. In some embodiments, the microfluidic structure of the wearable sensing device is present as a module of an article of clothing, e.g., a t-shirt. In some embodiments, the wearable sensing device is used to measure the concentration of at least one of Ca²⁺, H⁺, K⁺, Na⁺, NH₄⁺, Cl⁻, NO₃⁻, NO₂⁻, and Mg²⁺ in sweat. In some embodiments, the wearable sensing device is electrically connected to electronic transducers and displays for data readouts. For example, an Arduino® based (4 cm×1.5 cm×0.3 cm) wearable potentiometric transducer for data processing and wireless transfer was developed herein. Due to the low cost, standardization, and small size of the Arduino board, its marriage with the fabric-based wearable sensing device enhances the wide application of wearable sensors to promote human healthcare.

In a first aspect, a method of introducing a microfluidic pattern to fabric is described, said method comprising:
introducing a pattern onto a film comprising a polymer;
positioning the film onto a first side of the fabric to produce a polymer film-fabric stack; and
exposing the polymer film-fabric stack to a solvent vapor to dissolve the polymer, wherein the dissolved polymer diffuses into the fabric to form hydrophobic areas in and/or on the fabric that define the microfluidic pattern.

In some embodiments, the method of the first aspect yields a fabric having a microfluidic pattern that is substantially similar to the pattern of the film comprising the polymer. In some embodiments, the microfluidic pattern comprises at least one reservoir and a reservoir channel connected to the reservoir. In some embodiments, the microfluidic pattern comprises one reservoir and a reservoir channel connected to the reservoir. In some other embodiments, the microfluidic pattern comprises two reservoirs or three reservoirs or four reservoirs or five reservoirs or more, each with a reservoir channel connected to the reservoir, wherein each reservoir channel is connected to a merging channel. In some embodiments, each reservoir channel facilitates directional delivery of sweat to the reservoirs(s) via capillary actions. In some embodiments, when more than one reservoir is present, the merging channel feeds the reservoir channels substantially equally. In some embodiments, the reservoir has a substantially oval shape, a substantially circular shape, or a substantially elliptical shape. It should be appreciated by the skilled artisan that when there are two or more reservoirs, the different reservoirs can have the same or different shapes. It should also be appreciated by the skilled artisan that when there are two or more reservoirs, the different reservoirs can have the same or different volumes. In some embodiments, the reservoir channels in the microfluidic pattern have a width in a range of about 400 μm to about 1000 μm, or about 600 μm to about 1000 μm, or about 700 μm to about 900 μm. It should be appreciated by the skilled artisan that when there are two or more reservoirs, and hence two or more reservoir channels, the different reservoir channels can have the same or different widths.

Regarding the polymer, the polymer material is chosen to infiltrate a microfluidic pattern into the fabric matrix. The polymers chosen can be dissolved by relative solvents. In some embodiments, the polymer comprises ABS. In some embodiments, the polymer comprises poly vinyl chloride (PVC). In some embodiments, the polymer comprises a mixture of polymer species. Other polymers are well within the knowledge of the skilled artisan. In some embodiments, a thickness of the film comprising polymer is about 0.01 mm to about 0.5 mm, or about 0.02 mm to about 0.2 mm, or about 0.05 mm to about 0.15 mm, or about 0.06 mm to about 0.1 mm. In some embodiments, a thickness of the fabric is in a range from about 0.1 mm to about 2 mm, or about 0.1 mm to about 1 mm or about 0.1 mm to about 0.5 mm. It should be appreciated by the person skilled in the art that the thicker the fabric, the thicker the film comprising polymer that should be used to ensure substantially complete diffusion of the polymer throughout the thickness of the fabric.

In some embodiments, the solvent vapor is introduced by dampening a second side of the fabric with the solvent. In some embodiments, the solvent can be selected from acetone, tetrahydrofuran, chloroform, ethylene dichloride, esters, and ketones that are known to dissolve the chosen polymer. In some embodiments, the solvent comprises acetone. In some embodiments, the polymer comprises ABS and the solvent vapor comprises acetone. In some embodiments, the solvent comprises tetrahydrofuran. In some embodiments, the polymer comprises PVC and the solvent vapor comprises tetrahydrofuran. In some other embodiments, the solvent vapor is introduced to the fabric within an enclosed container that comprises a level of solvent vapors therein. In some embodiments, the polymer film-fabric stack is exposed to solvent vapor at temperature in a range from about 20° C. to about 30° C. or about room temperature. In some embodiments, the polymer film-fabric stack is exposed to solvent vapor at approximately atmospheric pressure. In some embodiments, the exposure time to solvent vapor is in a range of about 5 sec to about 20 sec when the thickness of the polymer film is in a range from about 0.06 mm to about 0.1 mm. In some embodiments, the ABS film-fabric stack is exposed to acetone vapor at temperature in a range from about 20° C. to about 30° C. or about room temperature. In some embodiments, the ABS film-fabric stack is exposed to acetone vapor at approximately atmospheric pressure. In some embodiments, the exposure time to acetone is in a range of about 5 sec to about 20 sec when the thickness of the ABS film is in a range from about 0.06 mm to about 0.1 mm. It should be appreciated that when the polymer film is thicker than 0.1 mm, the exposure time to solvent vapor may be in a range from about 20 sec to about 60 see to ensure substantially complete diffusion of the polymer throughout the thickness of the fabric matrix, as readily understood by the person skilled in the art.

In some embodiments, the fabric is selected from cotton, bamboo, polyester, linen, lyocell, modal, silk (natural, artificial, or synthetic spider), microfiber, satin, cellulose acetate, cellulose triacetate, rayon, polyester, nylon, viscose, hemp, wool, polypropylene, carbon fibers, lycra-spandex, thermoplastic polyurethane (TPU), polyamide, polyethylene (PE), polyethersulfone (PES), polyether-polyurea copolymers, or a blend of two or more of these materials.

In in embodiment of the first aspect, a method of introducing a microfluidic pattern to fabric comprises:
introducing a pattern onto a film comprising acrylonitrile butadiene styrene (ABS);
positioning the film onto a first side of the fabric to produce an ABS film-fabric stack; and
exposing the ABS film-fabric stack to acetone vapor to dissolve the ABS,
   wherein the dissolved ABS diffuses into the fabric to form hydrophobic areas in and/or on the fabric that define the microfluidic pattern.

After practicing the method of the first aspect herein to produce the microfluidic pattern on the fabric, the dissolved polymer, e.g., ABS, substantially infiltrates through the fabric matrix making the entire layer hydrophobic, while unaffected parts (meaning parts that did not have any polymer film positioned over the fabric) are fabric with no dissolved polymer and hence are substantially hydrophilic.

In a second aspect, an article of fabric comprising a hydrophobic microfluidic pattern is described, wherein the microfluidic pattern comprises at least one reservoir and a reservoir channel connected to the reservoir, and wherein the microfluidic pattern comprises a polymer.

In some embodiments, the polymer comprises ABS. In some embodiments, the polymer comprises poly vinyl chloride (PVC). Other polymers are well within the knowledge of the skilled artisan.

In some embodiments, the fabric is selected from cotton, bamboo, polyester, linen, lyocell, modal, silk (natural, artificial, or synthetic spider), microfiber, satin, cellulose acetate, cellulose triacetate, rayon, polyester, nylon, viscose, hemp, wool, polypropylene, carbon fibers, lycra-spandex, thermoplastic polyurethane (TPU), polyamide, polyethylene (PE), polyethersulfone (PES), polyether-polyurea copolymers, or a blend of two or more of these materials.

In some embodiments, the microfluidic pattern comprises at least one reservoir and a reservoir channel connected to the reservoir. In some embodiments, the microfluidic pattern comprises one reservoir and a reservoir channel connected to the reservoir. In some other embodiments, the microfluidic pattern comprises two reservoirs or three reservoirs or four reservoirs or five reservoirs or more, each with a reservoir channel connected to the reservoir, wherein each reservoir channel is connected to a merging channel. In some embodiments, each reservoir channel facilitates directional delivery of sweat to the reservoirs(s) via capillary actions. In some embodiments, when more than one reservoir is present, the merging channel feeds the reservoir channels substantially equally. In some embodiments, the reservoir has a substantially oval shape, a substantially circular shape, or a substantially elliptical shape. It should be appreciated by the skilled artisan that when there are two or more reservoirs, the different reservoirs can have the same or different shapes. It should also be appreciated by the skilled artisan that when there are two or more reservoirs, the different reservoirs can have the same or different volumes. In some embodiments, the reservoir channels in the microfluidic pattern have a width in a range of about 400 μm to about 1000 μm, or about 600 μm to about 1000 μm, or about 700 μm to about 900 μm. It should be appreciated by the skilled artisan that when there are two or more reservoirs, and hence two or more reservoir channels, the different reservoir channels can have the same or different widths.

In some embodiments, the microfluidic pattern comprises at least two reservoirs and reservoir channels connected to each reservoir, wherein each reservoir channel is connected to a merging channel. In some embodiments, each reservoir further comprises at least one electrode. In some embodiments, at least one reservoir comprise an analyte species indicator electrode and one of the reservoirs comprises a reference electrode. In some embodiments, the analyte species are selected from Ca²⁺, H⁺, K⁺, Na⁺, NH₄+, Cl⁻, NO₃⁻, NO₂⁻, and Mg²⁺. In some embodiments, the analyte species indicator electrode comprises an analyte species ionophore specific to the analyte species to be measured or monitored. In some embodiments, the reference electrode is a Ag/AgCl electrode. In some embodiments, the microfluidic pattern comprises three, four or five reservoirs and reservoir channels connected to each reservoir, wherein each reservoir channel is connected to a merging channel. In some embodiments, each reservoir further comprises at least one electrode. In some embodiments, one of the reservoirs comprises a reference electrode and the other reservoirs each comprise an analyte species indicator electrode, wherein the analyte species indicator electrodes are the same as or different from one another.

In some embodiments of the second aspect, the article is flexible, which allows it to be placed in direct contact with skin so that the microfluidic pattern can direct the sweat and so that the electrodes can come in direct contact with the skin.

In some embodiments of the second aspect, the article of fabric comprises a hydrophobic microfluidic pattern, wherein the microfluidic pattern comprises at least one reservoir and a reservoir channel connected to the reservoir, and wherein the microfluidic pattern comprises ABS.

In some embodiments of the second aspect, the article of fabric comprises a hydrophobic microfluidic pattern, wherein the microfluidic pattern comprises at least two reservoirs and a reservoir channel connected to each reservoir, and wherein the microfluidic pattern comprises a polymer.

In some embodiments of the second aspect, the article of fabric comprises a hydrophobic microfluidic pattern, wherein the microfluidic pattern comprises at least two reservoirs and a reservoir channel connected to each reservoir, and wherein the microfluidic pattern comprises ABS.

In a third aspect, a wearable sensing device comprising the article of the second aspect is described.

In some embodiments, the wearable sensing device is attached to a user's skin using an adhesive material. In some embodiments, the adhesive layer reversibly adheres the wearable sensing device to the skin surface. In some embodiments, the adhesive layer comprises medical grade acrylic or medical grade silicon. In some embodiments, the wearable sensing device may be integrated into clothing (t-shirts, pants, undergarments, etc.) or accessories (e.g., gloves, socks, headbands, hats, etc.), for example, as a module, and held in place using, e.g., suction, adhesive material, a band, compression clothing, or regular or hook-and-loop tape. The wearable sensing device may be affixed to various portions of a user's body, including, but not limited to, the torso, legs, back, neck, arms, hands, etc. For example, in some embodiments, the wearable sensing device comprises a band that can encircle an arm or a leg and can be tightened (e.g., using hook-and-loop connecting means or elastic) to effectuate the direct contact of the article of the second aspect with the skin.

In some embodiments, the electrodes (i.e., at least one indicator electrode and reference electrode) and a potentiometer are connectable via a wire. For example, the electrodes can have attached thereto (e.g. soldered) a first end of a wire. The second end of the wire can have a connector, e.g., a mateable plug. The corresponding mate of the mateable plug can be attached to a wire that connects to the potentiometer. In some embodiments, an electronics module comprises the potentiometer. The electronics module can further comprise at least one of amplifiers (e.g., for amplifying signals), voltage reference circuits, and analog-to-digital converters (ADC)/multiplexer (MUX). Accordingly, in some embodiments, the electronics module can convert analyte measurements from analog to digital signals before transmitting those signals to a control module.

In some embodiments, the control module includes a memory device and a microcontroller (MCU). In some embodiments, the control module also includes a wireless communications unit (e.g., a Bluetooth unit), power management circuitry, and a battery unit. The battery unit may be rechargeable, for example, using induction charging. In some embodiments, the battery unit (or other components of wearable sensing device) may be powered by sweat (e.g., using salt-based batteries). Further, in some embodiments, to reduce power consumption, one or more components of the wearable sensing device may remain inactive until the presence of sweat is detected. In some embodiments, memory device may be a microSD card or other data storage device that is removably insertable into or permanently installed in the control module.

In some embodiments, the control module is capable of wireless communication with a remote computing device (e.g., a mobile computing device) using Bluetooth communications or alternative approaches such as direct Wi-Fi. In some embodiments, the control module is communicatively connected directly to a computing device, e.g., using a USB or a USC cable, or the like. In some embodiments, the control module is capable of bi-directional communication, allowing control module or the remote computing device to store and recall prior data in the event of a communications breakdown.

In some embodiments, the control module communicates with an associated software application installed on a mobile computing device. The software application may include multiple functions that assist a user in using the wearable sensing device. For example, the software application may assist a user in identifying the species to be analyzed and entering calibration data. The software application may also upload all data to a cloud storage system, include algorithms for analyzing trends in the data, and enable users to customize the data analysis. The software application may also enable data (in a raw form or subsequent to analysis and conversion into relevant concentration values) to be displayed on the mobile computing device. For example, in some embodiments, the software application (or control module itself) may calculate concentrations based on potentiometric measurements as sweat comes in contact with the electrodes described herein as described herein. IN some embodiments, data may be stored on the control module as a backup, and downloaded to mobile communications device upon re-establishment of communications.

In some embodiments, the electronics module and the control module are contained in the same unit, which has a small footprint and is low-weight and low-profile, improving device com-fort and wearability, and improving mechanical and electrical reliability of the unit during natural body movements because internal mechanical stresses are significantly reduced. In some embodiments, the unit is wearable and can comprise attachment means such that the unit can be wrapped to a user's arm or leg. For example, in some embodiments, the unit can be fastened on the user's arm by a sports loop band. In some other embodiments, the unit is attached to an apparatus (e.g., a running treadmill or an indoor bicycle), stored in a pocket of a garment (e.g., pants or shirt), or may be attached, for example, to a belt or a heart-rate monitor strap (e.g., using a clip or hook and loop fasteners) and the unit comprises a length of wire that is long enough to attach, via the mateable plugs, to the wire emanating from the electrodes of the wearable sensing device. In some embodiments where the wearable sensing device is incorporated into a garment, the wire may be a flexible conductor sewn or otherwise incorporated into the garment such that the mateable plug is located in an unobtrusive location for connection to the corresponding mateable plug associated with the wire from the unit.

In some embodiments, the wearable sensing device of the third aspect comprises: the article of the second aspect, wherein each reservoir comprises an electrode: an electronics module; and a control module, wherein the electrodes, the electronics module and the control module are communicatively connected to one another. In some embodiments, the communicative connection includes the use of wires. In some embodiments, the communicative connection is wireless.

In a fourth aspect, a method of quantitating an analyte species found in sweat of a subject in real time is described, said method comprising:
positioning at least one indicator electrode in a first reservoir and a reference electrode in a second reservoir of the article of fabric comprising a hydrophobic microfluidic pattern of the second aspect;
electrically connecting the electrodes to a potentiometer; and
receiving signals from the potentiometer as sweat contacts the electrodes, wherein an electronic response of the potentiometer changes as sweat contacts the electrodes; and
quantitating the amount of analyte species found in the sweat of the subject based on the signals received from the potentiometer.

In some embodiments, the article of the second aspect is present as the wearable sensing device of the third aspect, wherein the electrodes (e.g., at least one indicator electrode and reference electrode) and a potentiometer are connectable via a wire. In some embodiments, the potentiometer is part of the electronics module of the wearable sensing device, in some embodiments, the electronics module communicates with the control module.

In some embodiments, the subject is a human subject undergoing a diagnostic procedure, a therapeutic procedure, or a fitness activity.

In practice, the wearable sensing device is calibrated relative to the analyte species of interest, as understood by the person skilled in the art. The article comprising the reservoirs and electrodes is positioned in contact with skin of a subject and as the subject sweats, sweat moves to the reservoirs via the reservoir channels based on capillary action. The electrodes are in electronic communication with the potentiometer, and as sweat contacts the electrodes, electronic signals are generated at the potentiometer, and the modules of the wearable sensing device record, collect, and process the data and transfers same to a computing device. Using the modules of the wearable sensing device, or the equivalent thereof, an amount of at least one analyte species can be quantified, as understood by the person skilled in the art.

In a fifth aspect, a method of analyzing biofluid from a subject is described, the method comprising the steps of: contacting the article of the second aspect with a skin surface of a subject; and analyzing the biofluid from the skin surface. In some embodiments, the biofluid is sweat. In some embodiments, the biofluid is analyzed for at least one analyte species. In some embodiments, the at least one analyte species is selected from Ca²⁺, H⁺, K⁺, Na⁺, NH₄, Cl, NO;. NO₂, and Mg²⁺. In some embodiments, the subject is a human subject undergoing a diagnostic procedure, a therapeutic procedure, or a fitness activity. In some embodiments, the article of the second aspect is conformally contacted with the skin surface.

### Example

### Chemicals and Materials

Magnesium chloride (MgCl₂), poly(vinyl chloride) (PVC), bis(2-ethylhexyl) sebacate (DOS), sodium tetraphenylborate, calcium ionophore II, and tetrahydrofuran (THF) were purchased from MilliporeSigma (MO, US). Hydrochloric acid (HCl), potassium chloride (KCl), and acetone were purchased from Thermo Fisher Scientific@(MA, US). Sodium chloride (NaCl) and calcium chloride (CaCl₂)) were purchased from Alfa Aesar® (MA, US). Silver/silver chloride (Ag/AgCl) and carbon inks were purchased from Ercon Inc. (MA, US). Acrylonitrile butadiene styrene (ABS) and polystyrene were purchased from McMaster-Carr (IL, US). Microfluidic syringe pumps were obtained from New Era (NJ, US). Artificial sweat was purchased from Nano Chemazone (Canada). Cotton fabric and white cotton T-shirts were purchased from Walmart® (Ar, US, thickness=0.3 mm).

Development of Fabric-Based Microfluidics with ABS Film Printing

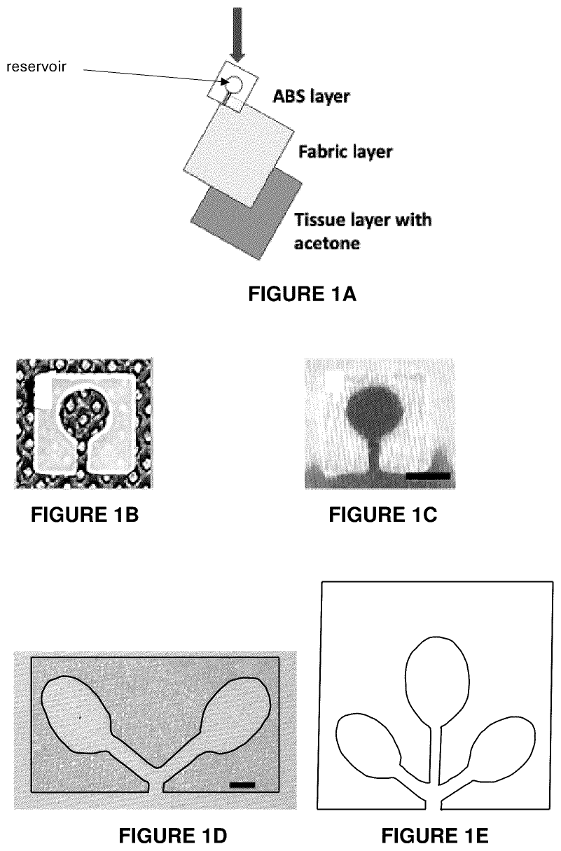

An ABS film (thickness=0.08 mm) was laser cut (raster power 100%, vector current 18%) to form the shape of the hydrophobic areas of the planned microfluidic device. Next, the ABS pattern was placed on top of a piece of cotton fabric. The ABS-fabric stack was then placed on top of a layer of paper towel soaked with acetone (see, e.g., FIG. 1A). The acetone needed to just dampen the paper towel without dried areas or dripping liquid. For example, 3 mL acetone was added to a square foot of paper towel. The acetone from the paper towel diffused up through the fabric layer to dissolve the ABS pattern on top of the stack, and the dissolved ABS then diffused down into the fabric to form the hydrophobic areas. Too long a treatment time would cause lateral smearing of the ABS compromising the microfluidic structure. Various fabrication times between 1 and 60 s were tested to determine the optimal time.

Microfluidics of various structures were designed. The single reservoir-channel design (see, FIG. 1B) was used to characterize the integrality of microfluidics under different acetone treatment times, the correction of the width of the channel between CAD design and, on fabric, and the simulations. The design with two reservoirs (FIG. 1D) was used to characterize flow rates and efficiency. The design with three reservoirs and a joint channel (FIG. 1E) was applied as the wearable microfluidic for sweat analyses. The three reservoirs were designed for the integration of two indicator electrodes and one reference electrode, respectively.

### Dimension Characterization of the Fabric-Based Microfluidics

Because the fabrication process involved acetone dissolving, the final channel size might differ from the size in the ABS pattern. To test if this was true, and if so, was there a correlation between the two dimensions, the channel sizes in the ABS patterns and the final microfluidic devices on the fabrics were measured. The channel width in an ABS pattern formed by a laser cutter was measured by a caliper. A channel was measured five times at random locations for one imaging. The channel width of the resulting fabric-based microfluidic device was measured under a calibrated optical microscope after adding a blue dye solution to the microfluidic for sharper color contrasts (see, FIG. 1C).

### Drainage Test of the Fabric-Based Microfluidics

A holder was 3D printed with a Fused Deposition Modeling (FDM) 3D printer and combined with Tygon tubing (inner diameter=1.5 mm). A reservoir on the fabric-based microfluidics was robustly attached to the tubing outlet of the holder without gaps using double-sided tape. A syringe pump with a needle (Gauge 16) was connected to the tube. A water-based dye solution (10 L) was added to a reservoir before the flow experiments. Then pure water from the syringe pump at different rates was delivered to the reservoir to wash out the dye at physiological sweating rates under various situations (inaction vs sports), including 3, 5, and 10 μL/min. An aliquot of 2 μL solution was taken using a pipet from the reservoir area at different time points. The sample was then mixed with 200 L DDI (doubly deionized) water in a 96-well plate. The absorbance at 630 nm of the diluted samples was measured by a plate reader (SpectraMax i3x, Molecular Devices). The initial pigment solution was defined as 100% concentrated, and a calibration curve was premade with dilutions of the initial solution. The percentage of the dye remaining in the reservoir area at different times was then calculated.

The shape of the reservoirs was optimized via COMSOL® simulations with the most efficient sweat delivery. In COMSOL®, cellulose was chosen as the substrate material with fluid speed scanning from 1 to 3 mm/min to mimic sweat passing the fabric layer. Also, a reservoir was designated as an inlet, and the end of the merging channel was designated as the outlet.

### Electrode Integration on the Fabric-Based Microfluidics

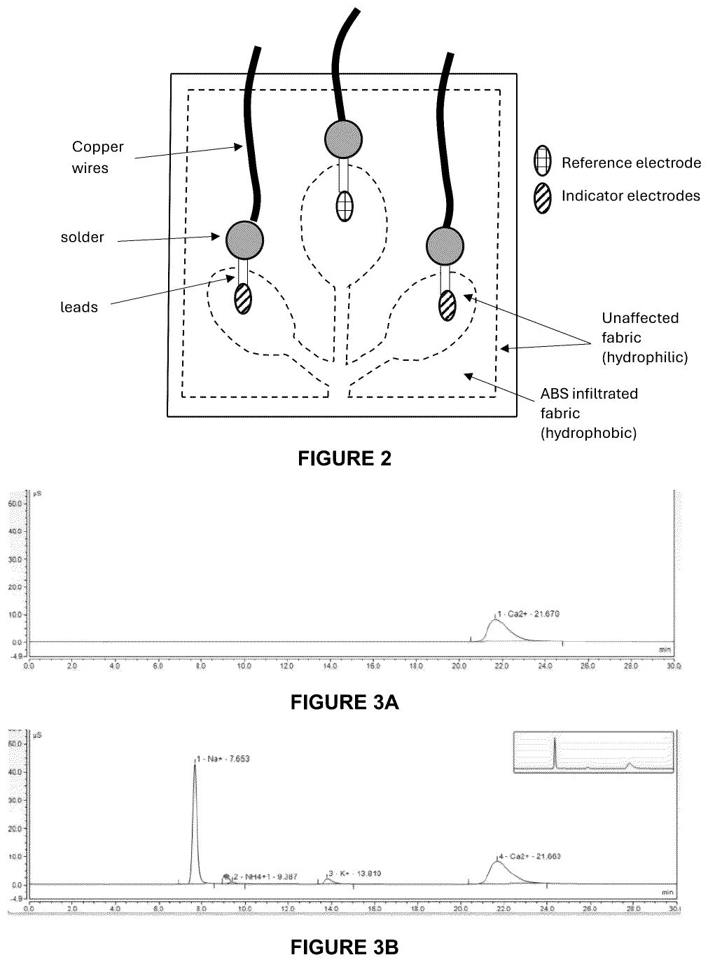

As shown in FIG. 2, three electrodes were screen-printed onto the three reservoirs of the microfluidic device. The two electrodes in the side reservoirs were indicator electrodes (duplicates), while the one in the middle served as the reference electrode. A printing stencil was designed in CorelDraw and laser cut from a polyester film (thickness=0.25 mm). Ag/AgCl printing ink was spread onto the stencil with a squeegee to screen-print electrodes onto the three reservoirs of the fabric microfluidics. The electrodes were 1 mm by 5 mm rectangles. After the Ag/AgCl ink was dried at 37° C. for 24 h, carbon ink was screen-printed onto the Ag/AgCl layer in the two side reservoirs as the electron transducing layer, followed by drying at room temperature overnight. To make a Ca²⁺ selective electrode, a Ca²⁺ membrane cocktail was made containing 1% Ca²⁺ ionophore II, 64.5% DOS, 33% poly(vinyl chloride), 0.5% sodium tetraphenylborate in THF (all concentrations in w/w). An aliquot of 5 μL of the Ca²⁺ membrane cocktail was then dropped onto the two indicator electrodes. Next, the electrodes were left in a fume hood for 12 h for the THF to dry out completely. The reference electrode was coated with a mixture made of polyvinyl butyral (PVB) saturated with NaCl. This mixture was made by dissolving 50 mg of NaCl and 79.1 mg PVB in 1 mL methanol. An aliquot of 3 μL of the PVB mixture was dropped onto the reference electrode, followed by air drying for 2 h, to provide constant [Cl⁻] around the reference electrode, and thus stable potential. The leads of the electrodes were connected to copper wires (diameter=0.5 mm) via soldering.

### The Arduino Program and Setup

The program was written in the free Arduino® IDE software. Arduino® Nano 33 board was applied as a connected part to a computer or a wearable device. The board in the wearable device could receive all initial data input from the fabric-based microfluidic sensor, process the data, and send the data wirelessly via low-energy Bluetooth to devices such as a smart phone. The App of Phyphox® was used to receive the data on an Android phone.

### Characterization of the Analytical Merits of the Wearable Sensor

### General Setup

Because it is known that the physiological concentration of Ca²⁺ in sweat is mainly between 0.2 mM and 2 mM, five Ca²⁺ standards were prepared in Ca-free artificial sweat at 0.125 mM, 0.25 mM, 0.5 mM, 1 mM, and 2 mM. The microfluidic wearable sensor with Ca²⁺ selective electrodes was connected to an Arduino® Nano 33 board via A0 and A6 (indicator electrodes) and GND port (reference electrode). Each standard solution was flown into the microfluidic reservoir at 3 μL/min for 40 s, starting from 0.125 mM to 2 mM. The voltage was read by Arduino® per second. The voltage readings from each standard solution were averaged and then plotted against the negative log of Ca²⁺ concentration (pCa) to generate a calibration curve.

### Selectivity Characterization

The possible interfering ions in sweat were tested in relevant/excessive amounts to determine the selectivity of the sensor for Ca²⁺. Five mixtures in water were prepared: 1 mM CaCl₂), 1 mM CaCl₂)+3 mM HCl, 1 mM CaCl₂)+5 mM KCl, 1 mM CaCl₂)+10 mM NaCl, and 1 mM CaCl₂)+0.25 mM MgCl₂ (the physiological concentrations of H⁺, K⁺, Na⁺, and Mg²⁺ in sweat are known to be around 3 mM, 1 mM, 10 mM, and 0.1 mM, respectively). Each solution was flown to the reservoir at 3 μL/min for 30 s and then switched to the next one with autonomous readings by the programmed Arduino® every 1 s.

### Accuracy Characterization

Two standard Ca²⁺ solutions in calcium-free artificial sweat (0.5 mM and 1.0 mM) were prepared by a first inventor and given to a second inventor for analytical recovery without telling disclosure of the concentrations (blind). A calibration curve was obtained by the second inventor as described above, which was used to calculate the concentrations of the “unknowns.” The tested and true values were then compared.

### Reproducibility Characterization

The measurements above (calibration curve, selectivity tests, and unknown recovery) were conducted on >10 devices fabricated from different batches on different days. All data were compared and pooled together so that error bars could tell the reproducibility.

### Near Real-Time Ca²⁺ Sensing on the Human Surface

### Standalone Measurement

A fabric-based microfluidic sensor was attached to the arm of a human via surgical tape. The Arduino® nano board was connected to the microfluidic device through soldered wires for voltage reading and transducing in near real-time (5 s per measurement). The board was fastened on the wearer's arm by a sports loop band. The Ca²⁺ in sweat was monitored for 25 min. Sweat around the sensor area was sampled by a pipet (10 L) every 6 min for ion chromatography measurements of Ca²⁺, as validations of the sensor results.

### Worn as a Part of a T-Shirt

The sleeve part of a T-shirt was laser cut to form a square hole of the same size as the fabric-based microfluidic device. The sensor was placed in the hole with the edges joined by narrow tape strips. A similar Arduino® board as described above was applied, but with the program wirelessly sending processed data to a phone in near real-time. The same sampling method was used to collect sweat in the sensor area for result validation.

### Ion-Chromatography Measurements of Ca²⁺ in the Collected Sweat

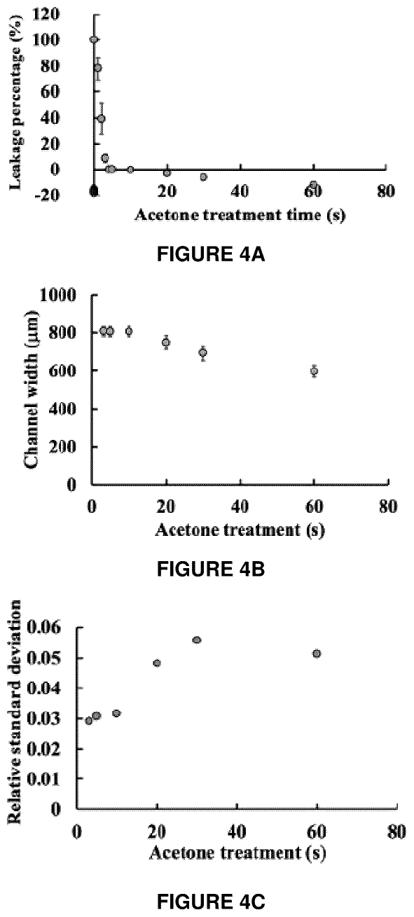

The Ca²⁺ content in sweat was measured via ion chromatography with a Thermo Scientific Dionex Integrion instrument. Ca²⁺ was separated along an IonPac CS16 column using reagent-free ion chromatography with a water mobile phase consisting of 30 mM sulfuric acid. Due to the low sample volume, 10 μL of each sweat sample was spiked into 5 mL standard Ca²⁺ solutions (0.5 mM) and submitted for IC measurement. Then, the Ca²⁺ content in sweat was calculated from the difference of the before- and after-spike standards. FIG. 3 shows an example chromatograph of standard 0.5 mM Ca²⁺ with and without the 10 μL sweat sample. The calibration range for this measurement was 0 to 1.25 mM (the five standards were 0.25 mM, 0.5 mM, 0.75 mM, 1 mM, and 1.25 mM) and the regression coefficients of determination (R²) were greater than 0.99.

### Data Analysis and Statistics

The t test and ANOVA were applied to compare data groups, and a significant difference was confirmed only when p values were smaller than 0.05.

### Results and Discussion

### The Novel Fabric-Based Microfluidics

To develop microfluidics on fabrics for sweat delivery, the hydrophobic areas that will define fluidic channels and reservoirs are an important step. Paraffin wax, although commonly used to make paper-based microfluidics, cannot be used for this purpose due to the high meltability at body surface temperature. While photoresists might be applicable, the process is tedious with large amounts of organic wastes (solvents) produced. Advantageously though, acrylonitrile butadiene styrene (ABS) is inexpensive, is low allergenic material, and dissolves in common solvents, such as acetone, and thus can be easily manipulated to diffuse through a porous substrate (e.g., fabrics) to form hydrophobic blocking areas. Although hot-pressing/lamination is widely used to combine ABS films onto a substrate, it was confirmed that this method is not sufficient to full melt the ABS, nor infiltrate the ABS into the fabrics. A new method is described herein using flexible ABS films assisted with acetone in a sandwich. Having a uniform layer of patterned ABS, like an ABS film, on top of a piece of fabric, with well-controlled infiltration, produces precise and reproducible microfluidic structures. For example, as illustrated in FIG. 1A, a microfluidic pattern was formed on an ABS film via laser cutting, with the void parts being channels and reservoirs in the resulting fabric microfluidics. This ABS pattern was placed on a piece of cotton fabric sitting on paper towel saturated with acetone. At room temperature, acetone liquid and its vapor could diffuse up through the cotton fabric layer to dissolve the ABS on top, which would then diffuse down through the fabric to form hydrophobic areas. FIG. 1B illustrates the microfluidic pattern comprising a single reservoir cut from an ABS film. After practicing the method described herein to produce the microfluidic structure, and although not shown herein, the dissolved ABS completely infiltrates through the fabric layer making the entire layer hydrophobic (as evidenced by an inability to pick up any hydrophilic blue dye), while unaffected parts were still cotton fabric and completely hydrophilic. As can be seen in FIG. 1C, after pipetting a blue food dye solution into the circular reservoir, the liquid could flow out of the channel without leakage out of the hydrophilic areas. FIG. 2 is an illustration of the ABS-printed microfluidic device, wherein the area within the dashed lines is the area infiltrated by the ABS and is hydrophobic and the rest of the fabric area is unaffected and hydrophilic.

The treatment time by the acetone vapor within the sandwich can affect the hydrophobicity of the microfluidic structure. Insufficient ABS dissolving and/or infiltration, observed if an acetone vapor treatment time was too short, would not form a tight hydrophobic seal through the fabric. On the other hand, too much acetone vapor treatment time would cause an overflow of the dissolved ABS laterally, compromising the channel and reservoirs (e.g., causing shrunk channels). Treatment times between 0 and 60 s were tested using the microfluidic model of FIG. 1B (i.e., comprising a single reservoir). After adding the blue dye solution to the reservoir and letting it flow out of the channel, the blue area (A_{b}) was measured in ImageJ. The designed area of the channel and reservoir was Ad (measured from the precut ABS pattern). The term “leakage percentage” was defined using Eq. (1) below. If the blue area was larger than the designed area, or a leakage percentage higher than 0, it meant the solution leaked out of the reservoir/channel. If the blue area was smaller than the designed area, with the leakage percentage being negative, it suggested ABS lateral diffusion shrinking the hydrophilic areas.

$$Leakage percentage = \frac{( Ab - Ad )}{Ad} \times 100 \% \qquad ( 1 )$$

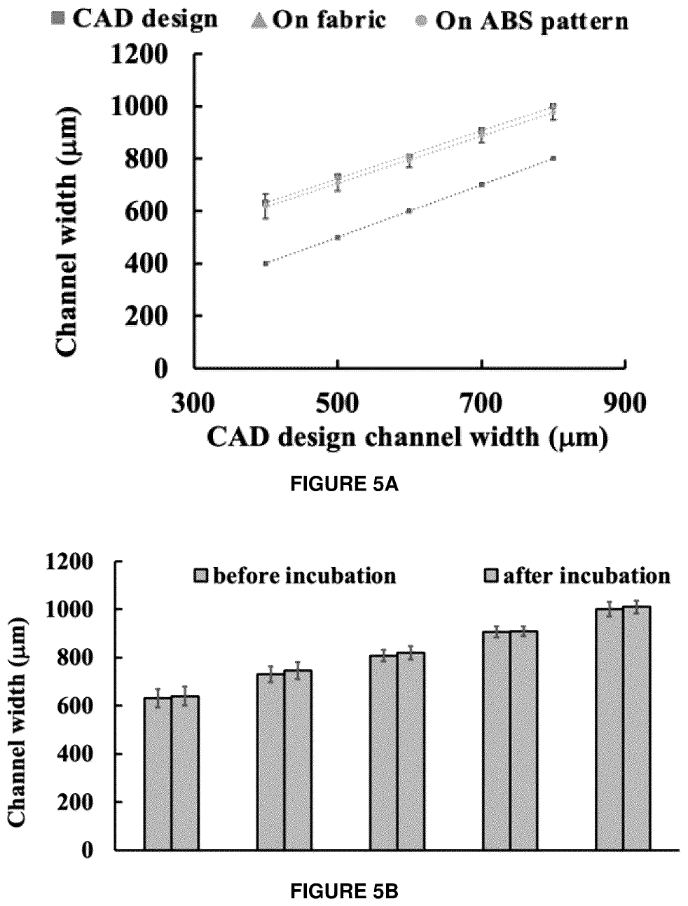

As shown in FIG. 4A, with a treatment shorter than 5 s, significant leakage was observed. Between 5 and 20 s, a leakage percentage of 0 was achieved, indicating perfect infusion of the ABS. When the treatment was longer than 20 s, the leakage percentage was negative, indicating undesired lateral ABS smearing. FIG. 4B further supported these observations based on channel width measurements of the resulting fabric-based microfluidics. Also, with too long treatment, the width variance of the same channel drastically increased (FIG. 4C). Based on these findings, it was determined that the optimal treatment time was 10 s for the present example.

ABS film having a thickness of 0.08 mm was used, the thinnest found to be commercially available. The cotton fabric was directly cut from a regular white T-shirt (0.3 mm thick), which was sufficient for the purpose of clothes-based wearable sensing. It is understood by the person skilled in the art that if fabrics and ABS films having other physical properties are used, different optimal treatment times may apply, which can be determined by similar methods presented herein.

### Characterization and Optimization of the Microfluidic Feature

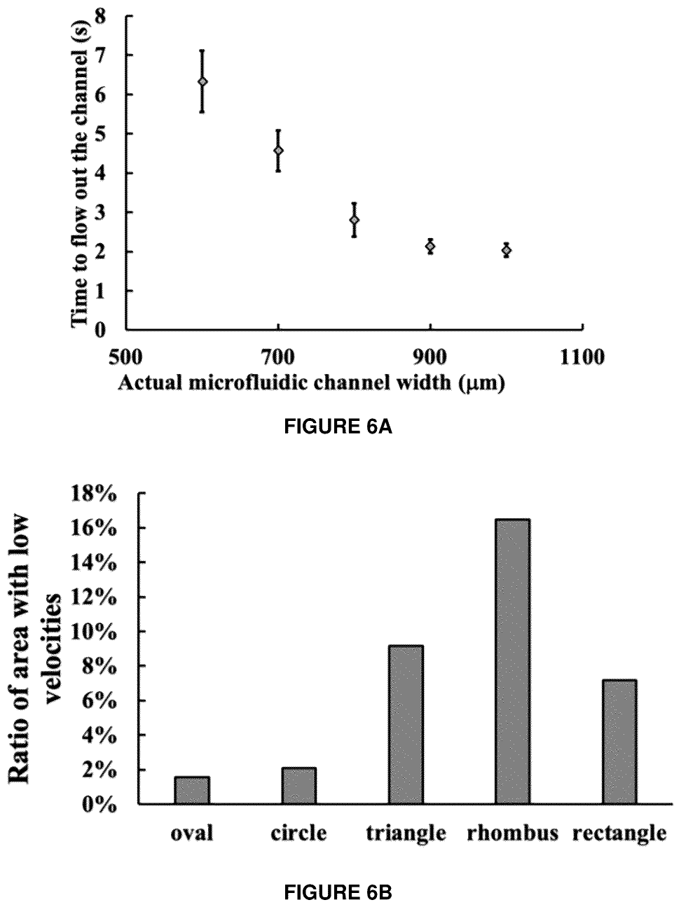

Because ABS is a thermal plastic, the cutting edges by a laser might melt more than expected. Therefore, the channel width designed in CAD, and the resulting channel width after cutting the CAD design through the ABS film, were compared. As shown in FIG. 5A, the squares are the designed channel widths in CAD (400, 500, 600, 700, 800 μm, respectively) and the circles are the channel widths in the cut ABS patterns. The cut ABS widths were significantly higher than the designs, likely caused by the overmelting of the ABS along the cutting traces. Nonetheless, this size change was reproducible and calibratable. The actual size on the ABS film was ˜200 μm larger than the design for all the widths tested. Moreover, the channel size in the ABS pattern was nearly the same as the channel width on fabric after the ABS film printing for 10 s, as shown in the triangles. Bright field microscopic views of the channels filled with the blue dye solution verified that no leakage was observed (not shown). The fabric-based microfluidics were incubated at 37° C. for 72 h and no ABS melting or smearing was observed that could compromise the microfluidic structures (FIG. 5B), which validated the long-term wearability of the platform. Because our laser-cutter cannot process cuttings smaller than 400 μm through ABS, also considering the 200 μm enlargement of a cut pattern than its CAD design, channels of 600 μm was the smallest that could be achieved.

### Optimize the Channel Size and Reservoir Shapes

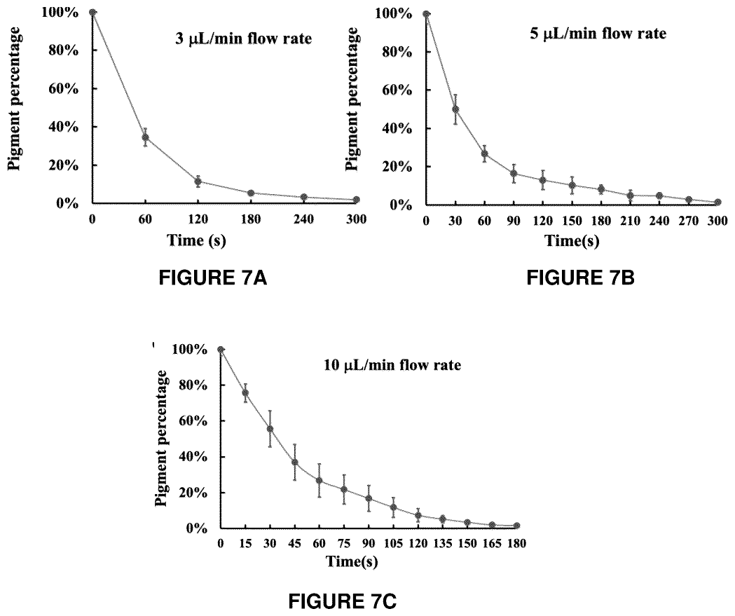

One reason to include microfluidic features on the wearable sensor was for efficient sweat delivery and thus sample refreshing in the detection zone. Human sweat glands are comprised of a secretory coil where sweat is initially produced and a dermal duct that pumps sweat via the epidermis to the surface with a natural pressure of ˜70 kPa. With the microfluidic reservoirs collecting sweat pumped out of the glands, the microfluidic channel could facilitate directional delivery of sweat via capillary actions. With a smaller channel, the capillary action force is larger, but the flow flux is smaller, and vice versa. Hence, the liquid delivery efficiency of various channel widths on the fabric were experimentally compared. As shown in FIG. 6A, with increased widths from 600 to 800 μm, the volumetric flow rate was faster (shortener drainage time), and after 800 in, the flow rates were no longer affected by the channel width (channel length=2.5 mm). For the purposes of the present example, the channel width of 800 μm was determined to be optimal, and was used in subsequent studies.

To find the most suitable reservoir shape, COMSOL® Multiphysics was used to simulate the fluids in the fabric-based microfluidic. Flow-stagnancy areas in a microfluidic reservoir, especially for wearable sensor detection, would ideally be as small as possible, providing efficient washing and thus accurate results for continuous near real-time detections. Velocity fields in the following reservoir shapes were tested: rectangular, triangle, circular, rhombus, and oval. The ratio of areas with the lowest velocities (<15% of the gross average) was calculated based on the various shapes (FIG. 6B). The oval (long axis parallel to the channel) showed the least low-velocity areas and thus the most homogeneity, which was thus determined as the optimal reservoir shape for subsequent applications in this example.

### Sweat Delivery Efficiency Validation

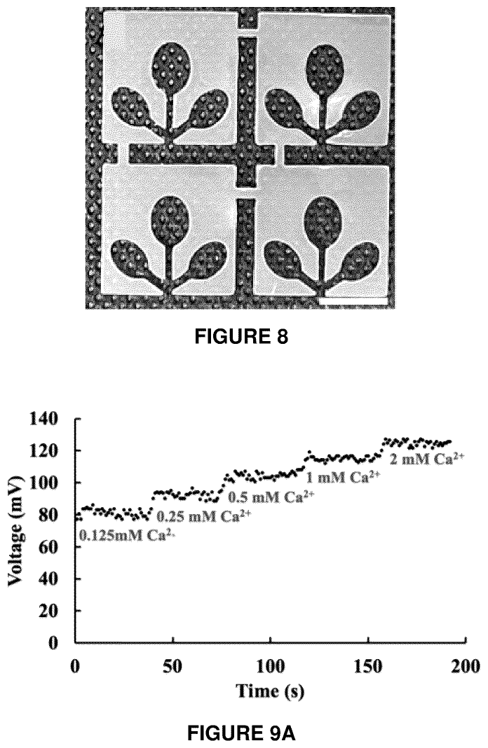

Based on the optimization above, fabric-based microfluidic devices were fabricated having two oval reservoirs and 800 μm wide channels, as shown in FIG. 1D. Water-based blue dye solution (10 μL) was pipetted onto the reservoir and was allowed to air dry. A 3D-printed holder was created that the microfluidic device could be taped onto. The holder could be attached to tubing that could be connected to a syringe, with the outlet of the tubing touching the reservoir of the microfluidic device. The dye-saturated reservoir was then washed with artificial sweat at 3, 5, and 10 μL/min, respectively. These flow rates reflected physiological sweating rates. The residue pigment in the reservoir was detected by absorbance spectroscopy (630 nm) in time courses. At the flow rates of 3 μL/min and 5 L/min, less than 5% of the blue dye remained in the reservoir after 5 min (FIGS. 7A and 7B). With L/min, >95% of the dye was washed out within 2 min (FIG. 7C). These results validated the efficient turnover of liquid samples in the microfluidic reservoirs.

### The Analytical Merits of the Sensor

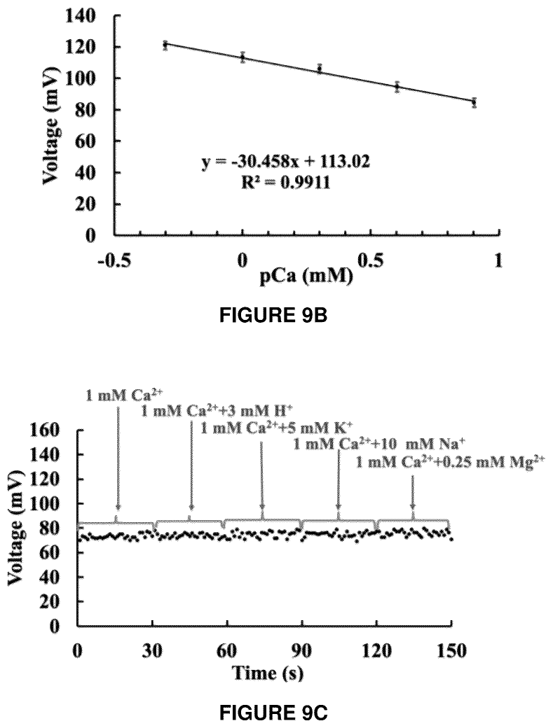

Ca²⁺ selective electrodes were screen-printed onto the fabric-based microfluidics to validate applicability. Ca²⁺ was chosen because this ion is an important participant in metabolism and mineral homeostasis. Unusual changes in Ca²⁺ concentrations can indicate possible diseases such as cirrhosis, renal failure, and myeloma (Roberson, 1981). The ABS film printing technology described herein can be easily scaled up for simultaneous multidevice fabrication. For example, multiple patterns on an ABS film, with each microfluidic device containing three oval reservoirs connected by channels of 800 μm width, is readily achievable (see, FIG. 8). As shown in FIG. 2, the two side reservoirs had Ca²⁺ indicator electrodes while the middle one had the reference electrode. The surface of the Ca²⁺ selective electrodes is a layer of PVC premixed with the Ca²⁺ ionophore, N,N,N′,N′-Tetracyclohexyl-3-oxapentanediamide, which has a hydrophilic pocket that forms a binding site specific for a Ca²⁺ (Nyein, 2016), creating a cross-membrane potential as a function of Ca²⁺ amounts. The two indicator electrodes were identical for duplicate measurements in each run. Wires were soldered onto the leads of the printed electrodes for conduction. For the purposes of this example, an Arduino® Nano board was used because of the small size, which is ideal for wearable applications.

The analytical merits of the fabric-based microfluidic sensor for Ca²⁺ quantitation was first characterized. The indicator electrodes were connected to the A0 and A6 port, and the reference electrode to the GND port of the Arduino® board. The board was connected to a laptop via a USB cable. A coding was developed to measure the potential differences between the indicator and reference electrodes. Potentiometric voltages were typically in the range of tens to hundreds of mV, which can be accurately detected by Arduino® without signal amplification.

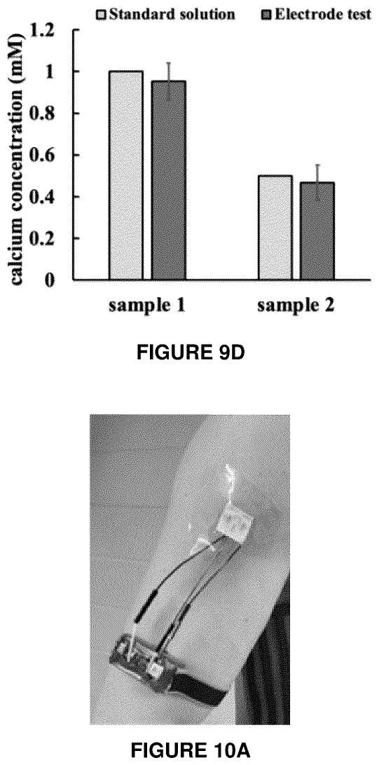

FIG. 9A shows near real-time measurements of Ca²⁺ standards (in artificial sweat) of 0.125 mM to 2 mM, flown through the sensor as described herein including the 3D-printed holder, tubing and a syringe pump setup. This concentration range covers physiological Ca²⁺ content in sweat (Nyein, 2016). With increased Ca²⁺, higher voltages were detected, in a linear Nernst way as shown in FIG. 9B. The limit of detection (LOD) was determined to be 5.8 μM, consistent with most published works focusing on Ca²⁺ measurements, which were in the single digit μM range. This is sufficient for wearable Ca²⁺ detection because the physiological range in sweat is mainly around 0.2 mM to 2 mM.

Selectivity can be an issue in potentiometric measurements. To validate the high selectivity toward Ca²⁺ of the sensor described herein, common cations in large amounts in sweat were tested, including H⁺, K⁺, Na⁺, and Mg²⁺. As indicated in FIG. 9C, these ions (comparable to physiological amount) did not interfere with the Ca²⁺ sensing. To validate the accuracy of the sensor, two Ca²⁺ standards were made by a first inventor and given to a second inventor blindly for concentration recovery. As shown in FIG. 9D, the detected concentrations were not significantly different from the recipe concentrations, suggesting accurate measurements by the sensor. Note that the data in FIGS. 9B and 9D were the average of multiple (>10) microfluidic sensors. The small error bars (especially on the calibration curve) confirmed the reproducibility of the sensor system.

### Apply the Sensor on Human Surface for Wireless Ca²⁺ Sensing

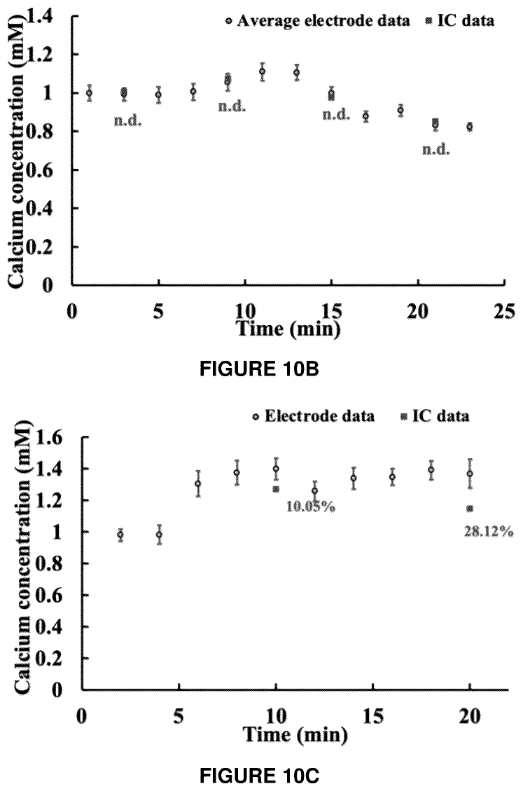

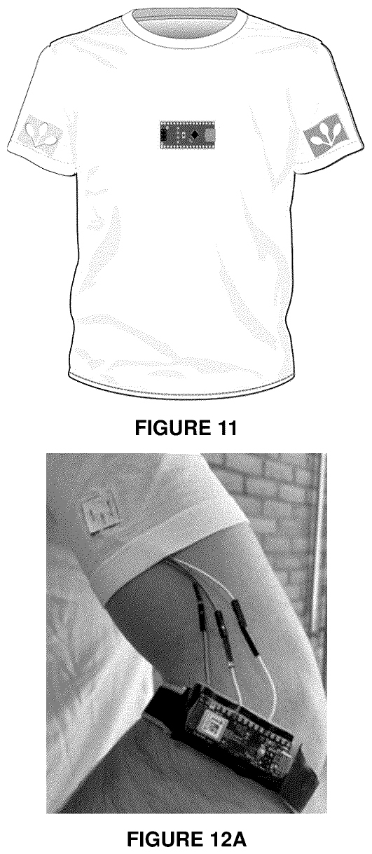

The fabric-based microfluidic sensor described herein was worn on the arm of the tester via surgical tape (see, FIG. 1A). The sensor was coupled to an Arduino® Nano board which was placed in a 3D printed holder fastened to the arm by a sport band. Two lithium coin batteries (CR2032) of 3.3 V were included in the holder to power the system. Specific coding was developed for data collection, recording, and wireless transfer to a cell phone. Near real-time wireless [Ca²⁺] receiving was collected on the free App of Phyphox. FIG. 10B shows the plotted results. During the 25 min monitoring, the concentration of Ca²⁺ was averaged between the two working electrodes every 2 min (5 s per reading, 24 readings within 2 min were averaged), and shown as open circles. The data showed a peak concentration between 10 and 15 min, followed by a gradual decrease. The closed squares are ion-chromatography analyses of Ca²⁺ from sweat sampled near the worn sensor at the designated time points, which were highly consistent with the sensor results without significant differences, strongly indicating the high accuracy of the sensor system described herein. Exercise will first induce increased electrolyte concentrations in sweat due to dehydration (Gao, 2016; Morgan, 2004). Then, vigorous exercise leads to parathyroid glands to release parathyroid hormone release, which stimulates bone resorption of free Ca²⁺, and thus reduced [Ca²⁺](Kohrt, 2018). These physiological facts were consistent with the quantitations recorded by the fabric-based microfluidic sensor.

A control sensor without the microfluidic structure was also tested, only with electrodes screen-printed exactly the same way on fabric squares. As shown in FIG. 10C, significant discrepancies between the sensor data (open circles) and the ion-chromatography data (closed squares) were produced. This discrepancy became more significant with time—at 10 min, a 10.5% difference was observed, while at 20 min, it was 28.12%. The sensor values were significantly higher than the real concentrations (closed squares, validated by ion chromatography). This was likely caused by insufficient sample refreshment at the electrode areas, where Ca²⁺ content kept accumulating. These data demonstrated the necessity to include microfluidics for efficient sample delivery in such sensors.

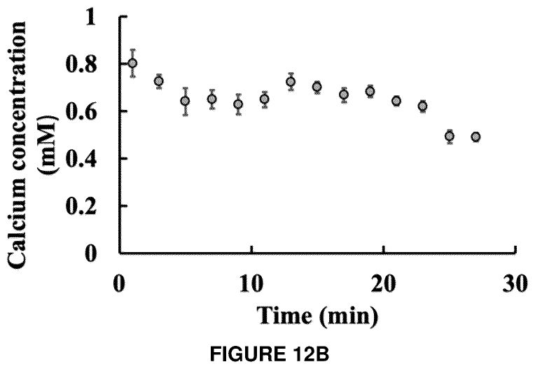

In addition to wearing the sensor in direct contact with your skin, the sensor can also be made to be a module of a T-shirt. As shown in FIG. 12A, a square was cut on the sleeve, which was covered by a plain fabric square when not used, via regular or hook-and-loop tape. Whenever needed, the plain fabric square was replaced by the fabric-based microfluidic device for near real-time detections and results read on a cell phone. We tested the scheme and found a similar trend of [Ca²⁺] in the sweat (FIG. 12B) as in FIG. 10B, with a peak between 10 and 15 min, followed by a slow decrease.

### CONCLUSION

A fabric-based microfluidic sensor platform was developed for wearable health monitoring. A new technology using ABS film printing was contributed, which can produce precise and reproducible fabric-based microfluidics with the potential of scaled mass-production. An Arduino-based potentiometer was also developed for wearable data collection, recording, processing, and wireless transfer. The simplicity of Arduino and the open-source nature of the related software will potentially facilitate the wide adoption of wearable sensing. The sensor system was validated by measuring [Ca²⁺] in sweat in near real-time, with accurate results generated. Meanwhile, the necessity of having microfluidic features for sweat delivery was also confirmed. The sensor can be attached directly to the skin of the user, or can be presented as a module of clothing, the success of which represents a critical step forward toward the ultimate goal of biochemically smart clothes.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Akther, F.; Yakob, S. B.; Nguyen, N.-T.; Ta, H. T. Surface Modification Techniques for Endothelial Cell Seeding in PDMS Microfluidic Devices. Biosensors 2020, 10 (11), 182, DOI: 10.3390/bios10110182
Altundemir, S.; Uguz, A. K.; Ulgen, K. A review on wax printed microfluidic paper-based devices for international health. Biomicrofluidics 2017, 11 (4), 041501, DOI: 10.1063/1.4991504
Anastasova, S.; Crewther, B.; Bembnowicz, P.; Curto, V.; Ip, H. M. D.; Rosa, B.; Yang, G.-Z. A wearable multisensing patch for continuous sweat monitoring. Biosens. Bioelectron. 2017, 93, 139-145, DOI: 10.1016/j.bios.2016.09.038
Bandodkar, A. J.; Wang, J. Non-invasive wearable electrochemical sensors: a review. Trends Biotechnol. 2014, 32 (7), 363-371, DOI: 10.1016/j.tibtech.2014.04.005
Bariya, M.; Nyein, H. Y. Y.; Javey, A. Wearable sweat sensors. Nat. Electron. 2018, 1 (3), 160-171, DOI: 10.1038/s41928-018-0043-y
Brueck, A.; Iftekhar, T.; Stannard, A. B.; Yelamarthi, K.; Kaya, T. A Real-Time Wireless Sweat Rate Measurement System for Physical Activity Monitoring. Sensors (Basel) 2018, 18 (2), 533, DOI: 10.3390/si8020533
Carrilho, E.; Phillips, S. T.; Vella, S. J.; Martinez, A. W.; Whitesides, G. M. Paper Microzone Plates. Anal. Chem. 2009, 81 (15), 5990-5998, DOI: 10.1021/ac9008473
Cincotto, F. H.; Fava, E. L.; Moraes, F. C.; Fatibello-Filho, O.; Faria, R. C. A new disposable microfluidic electrochemical paper-based device for the simultaneous determination of clinical biomarkers. Talanta 2019, 195, 62-68, DOI: 10.1016/j.talanta.2018.11.022
de Almeida Monteiro Melo Ferraz, M.; Nagashima, J. B.; Venzac, B.; Le Gac, S.; Songsasen, N. 3D printed mold leachates in PDMS microfluidic devices. Sci. Rep. 2020, 10 (1), 994, DOI: 10.1038/s41598-020-57816-y
Dungchai, W.; Chailapakul, O.; Henry, C. S. Electrochemical Detection for Paper-Based Microfluidics. Anal. Chem. 2009, 81 (14), 5821-5826, DOI: 10.1021/ac9007573
Dungchai, W.; Chailapakul, O.; Henry, C. S. A low-cost, simple, and rapid fabrication method for paper-based microfluidics using wax screen-printing. Analyst 2011, 136 (1), 77-82, DOI: 10.1039/COAN00406E
Emaminejad, S.; Gao, W.; Wu, E.; Davies, Z. A.; Yin Yin Nyein, H.; Challa, S.; Ryan, S. P.; Fahad, H. M.; Chen, K.; Shahpar, Z.; Talebi, S.; Milla, C.; Javey, A.; Davis, R. W. Autonomous sweat extraction and analysis applied to cystic fibrosis and glucose monitoring using a fully integrated wearable platform. Proc. Natl. Acad. Sci. U.S.A 2017, 114 (18), 4625-4630, DOI: 10.1073/pnas.1701740114
Farajikhah, S.; Cabot, J. M.; Innis, P. C.; Paull, B.; Wallace, G. Life-Saving Threads: Advances in Textile-Based Analytical Devices. ACS Comb. Sci. 2019, 21 (4), 229-240, DOI: 10.1021/acscombsci.8b00126
Gao, W.; Emaminejad, S.; Nyein, H. Y. Y.; Challa, S.; Chen, K.; Peck, A.; Fahad, H. M.; Ota, H.; Shiraki, H.; Kiriya, D.; Lien, D.-H.; Brooks, G. A.; Davis, R. W.; Javey, A. Fully integrated wearable sensor arrays for multiplexed in situ perspiration analysis. Nature 2016, 529 (7587), 509-514, DOI: 10.1038/nature16521.
Ghaffari, R.; Rogers, J. A.; Ray, T. R. Recent progress, challenges, and opportunities for wearable biochemical sensors for sweat analysis. Sens. Actuators, B 2021, 332, 129447, DOI: 10.1016/j.snb.2021.129447
Koh, A.; Kang, D.; Xue, Y.; Lee, S.; Pielak, R. M.; Kim, J.; Hwang, T.; Min, S.; Banks, A.; Bastien, P.; Manco, M. C.; Wang, L.; Ammann, K. R.; Jang, K.-I.; Won, P.; Han, S.; Ghaffari, R.; Paik, U.; Slepian, M. J.; Balooch, G.; Huang, Y.; Rogers, J. A. A soft, wearable microfluidic device for the capture, storage, and colorimetric sensing of sweat. Science Translational Medicine 2016, 8 (366), 366, DOI: 10.1126/scitranslmed.aaf2593
Kohrt, W. M.; Wherry, S. J.; Wolfe, P.; Sherk, V. D.; Wellington, T.; Swanson, C. M.; Weaver, C. M.; Boxer, R. S. Maintenance of Serum Ionized Calcium During Exercise Attenuates Parathyroid Hormone and Bone Resorption Responses. J. Bone Miner Res. 2018, 33 (7), 1326-1334, DOI: 10.1002/jbmr.3428.
Liu, M.; Zhang, C.; Liu, F. Understanding wax screen-printing: A novel patterning process for microfluidic cloth-based analytical devices. Anal. Chim. Acta 2015, 891, 234-246, DOI: 10.1016/j.aca.2015.06.034
Liu, J.; Kong, X.; Wang, H.; Zhang, Y.; Fan, Y. Roll-to-roll wax transfer for rapid and batch fabrication of paper-based microfluidics. Microfluid. Nanofluid. 2020, 24 (1), 6, DOI: 10.1007/s10404-019-2310-2
Liu, P.; Li, B.; Fu, L.; Huang, Y.; Man, M.; Qi, J.; Sun, X.; Kang, Q.; Shen, D.; Chen, L. Hybrid Three Dimensionally Printed Paper-Based Microfluidic Platform for Investigating a Cell's Apoptosis and Intracellular Cross-Talk. ACS Sens. 2020, 5 (2), 464-473, DOI: 10.1021/acssensors.9b02205
Martinez, A. W.; Phillips, S. T.; Whitesides, G. M. Three-dimensional microfluidic devices fabricated in layered paper and tape. Proc. Natl. Acad. Sci. U.S.A 2008, 105 (50), 19606-19611, DOI: 10.1073/pnas.0810903105
Martinez, A. W.; Phillips, S. T.; Whitesides, G. M.; Carrilho, E. Diagnostics for the Developing World: Microfluidic Paper-Based Analytical Devices. Anal. Chem. 2010, 82 (1), 3-10, DOI: 10.1021/ac9013989
Moonen, E. J. M.; Haakma, J. R.; Peri, E.; Pelssers, E.; Mischi, M.; den Toonder, J. M. J. Wearable sweat sensing for prolonged, semicontinuous, and nonobtrusive health monitoring. VIEW 2020, 1 (4), 20200077, DOI: 10.1002/VIW.20200077
Morbioli, G. G.; Speller, N. C.; Stockton, A. M. A practical guide to rapid-prototyping of PDMS-based microfluidic devices: A tutorial. Anal. Chim. Acta 2020, 1135, 150-174, DOI: 10.1016/j.aca.2020.09.013
Morgan, R.; Patterson, M.; Nimmo, M. Acute effects of dehydration on sweat composition in men during prolonged exercise in the heat. Acta Physiol. Scand. 2004, 182 (1), 37-43, DOI: 10.1111/j.1365-201X.2004.01305.x.
Nilghaz, A.; Liu, X.; Ma, L.; Huang, Q.; Lu, X. Development of fabric-based microfluidic devices by wax printing. Cellulose 2019, 26 (5), 3589-3599, DOI: 10.1007/s10570-019-02317-z
Nyein, H. Y. Y.; Gao, W.; Shahpar, Z.; Emaminejad, S.; Challa, S.; Chen, K.; Fahad, H. M.; Tai, L.-C.; Ota, H.; Davis, R. W.; Javey, A. A Wearable Electrochemical Platform for Noninvasive Simultaneous Monitoring of Ca2+ and pH. ACS Nano 2016, 10 (7), 7216-7224, DOI: 10.1021/acsnano.6b04005.
Prabhu, A.; Giri Nandagopal, M. S.; Peralam Yegneswaran, P.; Singhal, H. R.; Mani, N. K. Inkjet printing of paraffin on paper allowslow-cost point-of-care diagnostics for pathogenic fungi. Cellulose 2020, 27 (13), 7691-7701, DOI: 10.1007/s10570-020-03314-3
Raj M, K.; Chakraborty, S. PDMS microfluidics: A mini review. J. Appl. Polym. Sci. 2020, 137 (27), 48958, DOI: 10.1002/app.48958
Robertson, W. G.; Marshall, R. W. Ionized calcium in body fluids. Crit Rev. Clin Lab Sci. 1981, 15 (2), 85-125, DOI: 10.3109/10408368109105869.
Sempionatto, J. R.; Moon, J.-M.; Wang, J. Touch-Based Fingertip Blood-Free Reliable Glucose Monitoring: Personalized Data Processing for Predicting Blood Glucose Concentrations. ACS Sens. 2021, 6 (5), 1875-1883, DOI: 10.1021/acssensors.1c00139
Sim, D.; Brothers, M. C.; Slocik, J. M.; Islam, A. E.; Maruyama, B.; Grigsby, C. C.; Naik, R. R.; Kim, S. S. Biomarkers and Detection Platforms for Human Health and Performance Monitoring: A Review. Advanced Science 2022, 9 (7), 2104426, DOI: 10.1002/advs.202104426
Urbanski, J. P.; Thies, W.; Rhodes, C.; Amarasinghe, S.; Thorsen, T. Digital microfluidics using soft lithography. Lab Chip 2006, 6 (1), 96-104, DOI: 10.1039/B510127A
Vatansever, F.; Burtovyy, R.; Zdyrko, B.; Ramaratnam, K.; Andrukh, T.; Minko, S.; Owens, J. R.; Kornev, K. G.; Luzinov, I. Toward Fabric-Based Flexible Microfluidic Devices: Pointed Surface Modification for pH Sensitive Liquid Transport. ACS Appl. Mater. Interfaces 2012, 4 (9), 4541-4548, DOI: 10.1021/am3008664
Wang, Y.; Wang, X.; Lu, W.; Yuan, Q.; Zheng, Y.; Yao, B. A thin film polyethylene terephthalate (PET) electrochemical sensor for detection of glucose in sweat. Talanta 2019, 198, 86-92, DOI: 10.1016/j.talanta.2019.01.104
Wang, Z.; Shin, J.; Park, J.-H.; Lee, H.; Kim, D.-H.; Liu, H. Engineering Materials for Electrochemical Sweat Sensing. Adv. Funct. Mater. 2021, 31 (12), 2008130, DOI: 10.1002/adfm.202008130
Xiao, J.; Fan, C.; Xu, T.; Su, L.; Zhang, X. An electrochemical wearable sensor for levodopa quantification in sweat based on a metal-Organic framework/graphene oxide composite with integrated enzymes. Sens. Actuators, B 2022, 359, 131586, DOI: 10.1016/j.snb.2022.131586
Yang, P.; Wei, G.; Liu, A.; Huo, F.; Zhang, Z. A review of sampling, energy supply and intelligent monitoring for long-term sweat sensors. npj Flexible Electronics 2022, 6 (1), 33, DOI: 10.1038/s41528-022-00165-9
Zhang, J.; Song, R.; Zhao, X.; Fang, R.; Zhang, B.; Qian, W.; Zhang, J.; Liu, C.; He, D. Flexible Graphene-Assembled Film-Based Antenna for Wireless Wearable Sensor with Miniaturized Size and High Sensitivity. ACS Omega 2020, 5 (22), 12937-12943, DOI: 10.1021/acsomega.0c00263
Zhang, Y.; Duan, H.; Li, G.; Peng, M.; Ma, X.; Li, M.; Yan, S. Construction of liquid metal-based soft microfluidic sensors via soft lithography. J. Nanobiotechnol. 2022, 20 (1), 246, DOI: 10.1186/s12951-022-01471-0

What is claimed is:

1. A method of introducing a microfluidic pattern to fabric, said method comprising:
introducing a pattern onto a film comprising a polymer;
positioning the film onto a first side of the fabric to produce a polymer film-fabric stack; and
exposing the polymer film-fabric stack to a solvent vapor to dissolve the polymer, wherein the dissolved polymer diffuses into the fabric to form hydrophobic areas in and/or on the fabric that define the microfluidic pattern.

2. The method of claim 1, wherein the microfluidic pattern is substantially the same as the pattern of the film comprising polymer.

3. The method of claim 1, wherein the solvent vapor is introduced by dampening a second side of the fabric with solvent.

4. The method of claim 1, wherein the microfluidic pattern comprises at least one reservoir and a reservoir channel connected to the reservoir.

5. The method of claim 1, wherein the polymer comprises acrylonitrile butadiene styrene (ABS) or poly vinyl chloride (PVC).

6. The method of claim 4, wherein the at least one reservoir has a substantially oval shape, a substantially circular shape, or a substantially elliptical shape.

7. The method of claim 1, wherein the fabric is selected from cotton, bamboo, polyester, linen, lyocell, modal, silk (natural, artificial, or synthetic spider), microfiber, satin, cellulose acetate, cellulose triacetate, rayon, polyester, nylon, viscose, hemp, wool, polypropylene, carbon fibers, lycra-spandex, thermoplastic polyurethane (TPU), polyamide, polyethylene (PE), polyethersulfone (PES), polyether-polyurea copolymers, or a blend of two or more of these materials.

8. An article of fabric comprising a hydrophobic microfluidic pattern thereon, wherein the pattern comprises at least one reservoir and a reservoir channel connected to the reservoir, and wherein the hydrophobic microfluidic pattern comprises a polymer.

9. The article of claim 8, wherein the reservoir has a substantially oval shape, a substantially circular shape, or a substantially elliptical shape.

10. The article of claim 8, wherein the polymer comprises acrylonitrile butadiene styrene (ABS) or poly vinyl chloride (PVC).

11. The article of claim 8, wherein the at least one reservoir further comprises at least one electrode.

12. The article of claim 11, wherein at least one reservoir comprises an analyte species indicator electrode and one reservoir comprises a reference electrode.

13. The article of claim 12, wherein the analyte species are selected from Ca²⁺, H⁺, K⁺, Na⁺, NH₄⁺, Cl⁻, NO₃⁻, NO₂⁻, and Mg²⁺.

14. The article of claim 8, which is a part of a wearable sensing device.

15. The article of claim 14, wherein the wearable sensing device further comprises an electronics module and a control module.

16. A method of quantitating an analyte species found in sweat of a subject in real time, said method comprising:
positioning at least one indicator electrode in a first reservoir and a reference electrode in a second reservoir of the article of fabric comprising a hydrophobic microfluidic pattern of claim 8;
electrically connecting the electrodes to a potentiometer; and
receiving signals from the potentiometer as sweat contacts the electrodes, wherein an electronic response of the potentiometer changes as sweat contacts the electrodes; and
quantitating the amount of analyte species found in the sweat of the subject based on the signals received from the potentiometer.

17. The method of claim 16, wherein as the subject sweats, sweat moves to the reservoirs via the reservoir channels based on capillary action.

18. The method of claim 16, wherein the article and the potentiometer are part of a wearable sensing device.

19. The method of claim 18, wherein the wearable sensing device records, collects, and processes data and transfers same to a computing device.

20. The method of claim 19, wherein the wearable sensing device wirelessly transfers data to a computing device.
